(19) United States
(12) Patent Application Publication
(10) Pub. No.: US 2018/0098917 A1
(43) Pub. Date: Apr. 12, 2018

(54) JEJUNAL SYSTEM AND METHOD OF OPERATION

(71) Applicant: THE RESEARCH INSTITUTE AT NATIONWIDE CHILDREN'S HOSPITAL, Columbus, OH (US)

(72) Inventor: Ish Kumar Gulati, Hilliard, OH (US)

(21) Appl. No.: 15/573,623

(22) PCT Filed: May 12, 2016
(86) PCT No.: PCT/US2016/032098

§ 371 Date: Nov. 13, 2017

Related U.S. Application Data
(60) Provisional application No. 62/160,343, filed on May 12, 2015.

(51) Int. Cl. A61J 15/00

(52) U.S. Cl. CPC A61J 15/0069; A61M 39/20; A61J 15/0073; A61J 15/0042

(57) ABSTRACT

A jejunal system and apparatus are disclosed. The jejunal system includes a sheath having a proximal end and a distal end. A plurality of lumens extend through the sheath between the proximal end and the distal end. A first lumen of the plurality of lumens is provided with a first aperture set and a second aperture set. The first aperture set is in fluid communication with the second aperture set via the first lumen. The jejunal system is dimensioned and configured such that, when the system is inserted into a human body, the first aperture set is located in a small intestine of the human body, and the second aperture set is located in a stomach of the human body.

### CROSS REFERENCES TO RELATED APPLICATIONS

The following application claims priority under 35 U.S.C. § 119(e) to co-pending U.S. Provisional Patent Application Ser. No. 62/160,343 filed May 12, 2015 entitled JEJUNAL SYSTEM AND APPARATUS. The above-identified application is incorporated herein by reference in its entirety for all purposes.

### TECHNICAL FIELD

The present disclosure generally relates to the healthcare field and, more particularly, to a jejunal system and apparatus that provides nutrients, aspiration, and redirects fluids in patients.

### BACKGROUND

Gastrostomy is a surgical procedure that involves inserting a tube directly into a patient's stomach via an artificial opening. One end of the tube remains inside the stomach, and the other end of the tube extends from outside the stomach and the patient's body. Nutrients can be passed through the tube and directly into the stomach for patients who are unable to orally consume an appropriate amount of calories and/or nutrients to promote growth and/or sustain healthy body function. In some cases, there can be slow gastric motility/propulsion which may result in particular foods (e.g., milk) backing up through the artificial opening or refluxing into the patient's esophagus. In such cases, trans pyloric-jejunal feeding tubes (gastro-jejunal tubes) may be used.

A trans pyloric-jejunal feeding tube is typically extended into the patient's duoendum/jejunum where continuous feeds are directly transported to the small intestine. Some trans pyloric-jejunal feeding tubes include a double lumen. One of the lumens is placed in the stomach for feeding, venting, and decompression, while the other lumen is placed in the jejunum for feeding.

Trans pyloric-jejunal feeding tubes are frequently used to provide nutrients to sick infants. In this application, there are commonly complaints of stomach distension, feeding intolerance, feeding residuals, vomiting, and even aspiration, which can lead to death or severe comorbidities. It is believed that these complaints stem from the fact that, despite there being a gastric outlet, the amount of gastric contents is too great to be emptied. This may be due to the fact that one of the lumens partially obstructs the gastric outlet at the narrowest and most muscular part of the pyloric junction.

### SUMMARY

A jejunal system and methods of operation are disclosed. The jejunal system includes a sheath having a proximal end and a distal end. A plurality of lumens extend through the sheath between the proximal end and the distal end. A first lumen of the plurality of lumens is provided with a first aperture set and a second aperture set. The first aperture set is in fluid communication with the second aperture set via the first lumen. The jejunal system is dimensioned and configured such that, when the jejunal system is inserted into a human body, the first aperture set is located in a stomach of the human body, and the second aperture set is located in a small intestine of the human body.

The jejunal system is formed by advancing a plurality of lumens through a sheath, the plurality of lumens running between a proximal end of the sheath and a distal end of the sheath. A first aperture set and a second aperture set are dimensioned in a first lumen of the plurality of lumens, wherein the first aperture set is in fluid communication with the second aperture set via the first lumen. The jejunal system is dimensioned and configured such that, when the system is inserted into a human body, the first aperture set is located in a small intestine of the human body, and the second aperture set is located in a stomach of the human body.

A jejunal system includes a sheath having a proximal end and a distal end and a plurality of lumens extending through the sheath between the proximal end and the distal end. The plurality of lumens comprises a first lumen being provided with a first aperture set, a second aperture set and a third aperture set. The first lumen comprises a partition fluidly separating the first lumen into a first flow path and a second flow path, wherein the first aperture set and the second aperture set are in fluid communication with the first flow path, and the third aperture set is in fluid communication with the second flow path. A second lumen is provided with a fourth aperture set. Wherein the jejunal system is dimensioned and configured such that, when the system is inserted into a human body, the first aperture set and the fourth aperture set are located in a small intestine of the human body, and the second aperture set and the third aperture set are located in a stomach of the human body.

### BRIEF DESCRIPTION OF THE DRAWINGS

The foregoing and other features and advantages of the present disclosure will become apparent to one skilled in the art to which the present disclosure relates upon consideration of the following description of the invention with reference to the accompanying drawings, wherein like reference numerals, unless otherwise described refer to like parts throughout the drawings and in which:

FIG. 1A is a perspective view of a jejunal system according to a first embodiment of the present disclosure;

FIG. 1B is a magnified section view of FIG. 1 along section lines B-B;

FIG. 1C is a magnified section view of FIG. 1 along section lines C-C;

FIG. 2 is a plan view of the jejunal system in FIG. 1;

FIG. 3 is a partial detail view of portions of the jejunal system in FIG. 1 identified by areas A-C with portions of the sheath removed to show the lumens;

FIG. 4 is a perspective view of a jejunal system according to a second embodiment of the present disclosure;

FIG. 5 illustrates the jejunal system in FIG. 1 in a first application;

FIG. 6 illustrates the jejunal system in FIG. 1 in a second application; and

FIG. 7 illustrates the jejunal system in FIG. 1 in a second application in a magnified view.

### DETAILED DESCRIPTION

Referring now to the figures wherein like numbered features shown therein refer to like elements throughout unless otherwise noted. The present disclosure relates generally to the healthcare field and, more particularly, to a jejunal system and apparatus that provides nutrients, aspiration, and redirects solids and fluids in patients.

### Jejunal System

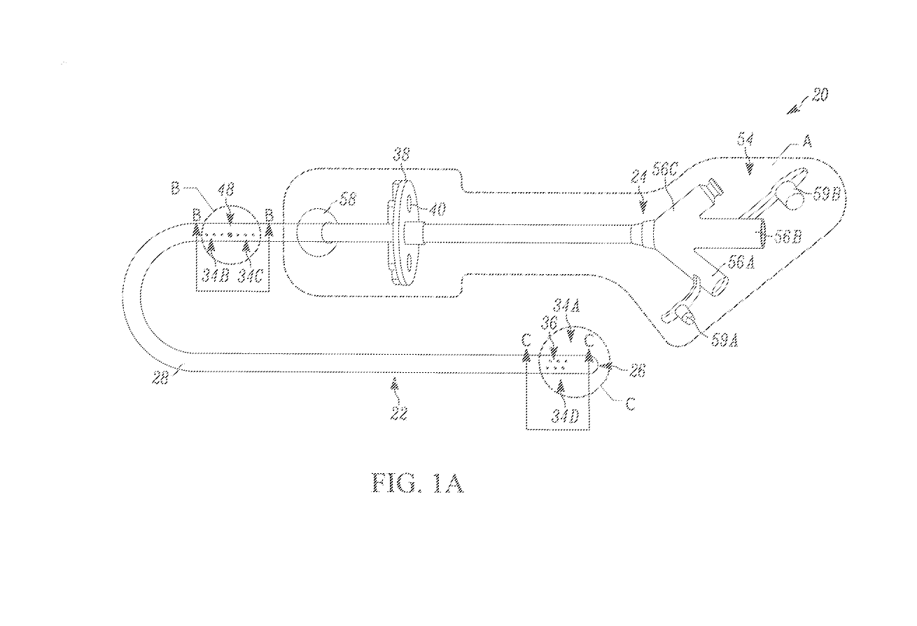

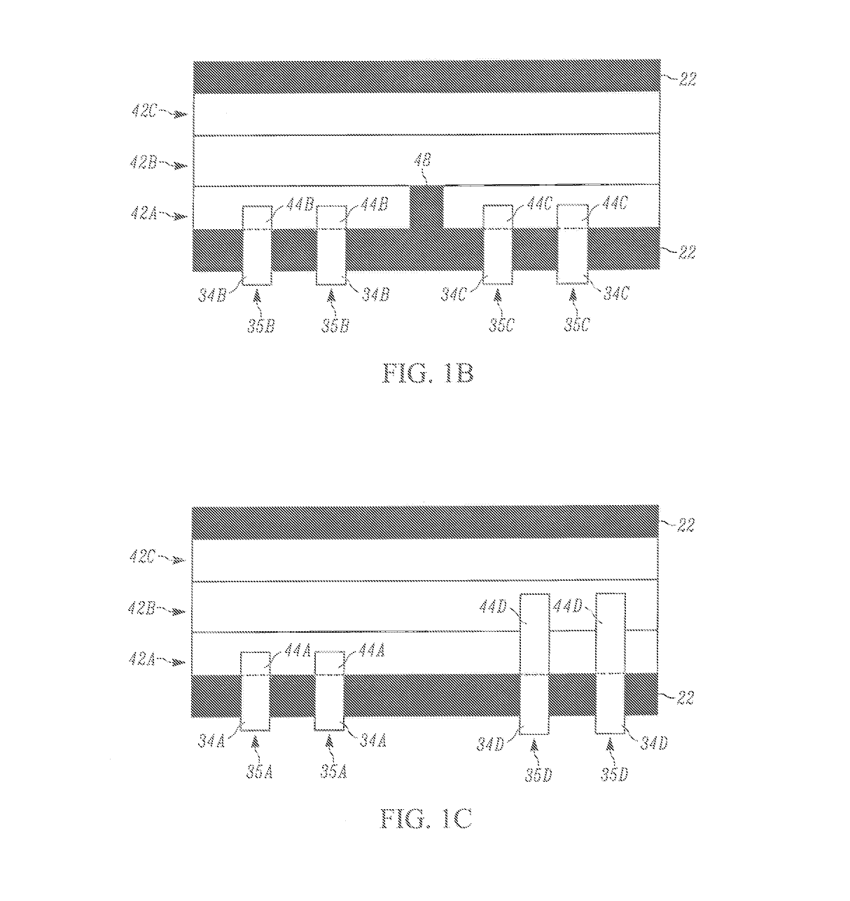

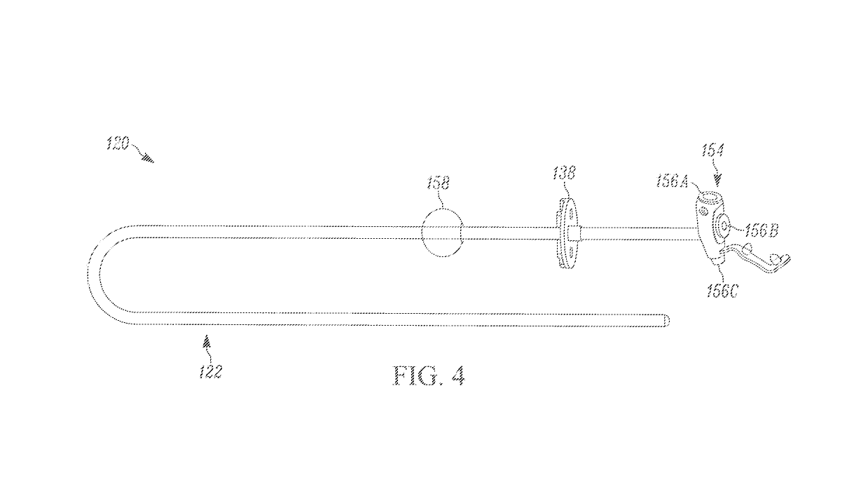

FIGS. 1-3 show a jejunal system 20 that includes a gastro-jejunal tube or naso-jejunal tube or apparatus according to one example embodiment of the present disclosure. The jejunal system 20 includes a sheath 22 that extends between a proximal end 24 and a distal end 26. The sheath 22 is substantially cylindrical shaped and includes an outer surface 28, illustrated in FIG. 1, and an inner surface 30, illustrated in FIG. 3, defining a passage. It will be appreciated that the sheath 22 can be any other suitable shape and in the illustrated example, embodiment is made from a flexible polymer such as plastic. A plurality of sheath aperture sets 34A-34D extend between the outer surface 28 and the inner surface 30 of the sheath 22. In one example embodiment, the sheath 22 can include four sheath aperture sets 34, with each sheath aperture set including three apertures 36. However, it is understood that the mouther of sheath aperture sets 34 may be increased or decreased, and the number of apertures 36 in each sheath aperture set 34 may likewise be increased or decreased. A retention collar 38 is secured to the outer surface 28. The retention collar 38 can have a circular shape and include attachment openings 40, however other configurations are possible. In the illustrated example embodiment, the retention collar 38 is made of rigid plastic, however other materials could be used of similar weight and strength.

A plurality of lumens 42A-42C extend through the sheath 22 between the proximal end 24 and the distal end 26, as illustrated in FIG. 3. In one example, the jejunal system 20 includes three lumens 42 formed from flexible tubing typically used in the medical field and compatible for inserting into patients. However, it is understood that the number of lumens 42 can be increased or decreased to tailor the jejunal system 20 for a particular application. The plurality of lumens 42A-42C include four lumen aperture sets 44A-44D, with each lumen aperture set 44 including three apertures 46. The number of lumen aperture sets 44 and apertures 46 may be increased or decreased, however it is preferable for the number of lumen aperture sets 44 and associated apertures 46 to be equal to the number of the sheath aperture sets 34 and associated apertures 36. In an example embodiment, the plurality of lumens 42A-42C comprise an internal lining comprising a non-stick coating, such as a highly hydrophobic or superhydrophobic non-stick coating. One such suitable internal lining is a product trademarked as LiquiGlide™, manufactured by LiquiGlide Inc. of Cambridge, Mass. and further disclosed in United States patent application publication numbers U.S. 2014/0314991 and U.S. 2014/0314975, the contents of both published applications are herein incorporated by reference.

The first and second lumen aperture sets 44A, 44B are provided on the first lumen 42A and are in fluid communication with one another via the first lumen 42A. The first and second lumen aperture sets 44A, 44B are located substantially adjacent the first and second sheath aperture sets 34A, 34B so as to be in fluid communication with the first and second sheath aperture sets 34A, 34B, respectively. In one example embodiment, the first sheath aperture set 34A, in conjunction with the first lumen aperture set 44A, forms a first aperture set 35A, as illustrated in FIG 1C. In one example embodiment the second sheath aperture set 34B, in conjunction with the second lumen aperture set 44B, forms a second aperture set 35B, as illustrated in FIG. 1B. The third lumen aperture set 44C is provided on the first lumen 42A and is located substantially adjacent the third sheath aperture set 34C so as to be in fluid communication with the third sheath aperture set 34C. In one example embodiment, the third sheath aperture set 34C, in conjunction with the third lumen aperture set 44C forms a third aperture set 35C, as illustrated in FIG. 1B. The third lumen aperture set 44C is fluidly divorced from the first and second lumen aperture sets 44A, 44B by a partition 48. The partition 48 is provided in the first lumen 42A and divides the first lumen 42A into a first flow path 50 and a second flow path 52. The first and second sheath aperture sets 34A, 34B are in fluid communication with the first flow path 50, and the third sheath aperture set 34C is in fluid communication with the second flow path 52. The fourth lumen aperture set 44D is provided on the second lumen 42B and is located substantially adjacent the fourth sheath aperture set 34D so as to be in fluid communication with the fourth sheath aperture set 34D. In one example embodiment, the fourth sheath aperture set 34D, in conjunction with the fourth lumen aperture set 44D, forms a fourth aperture set 35D, as illustrated in FIG. 1C.

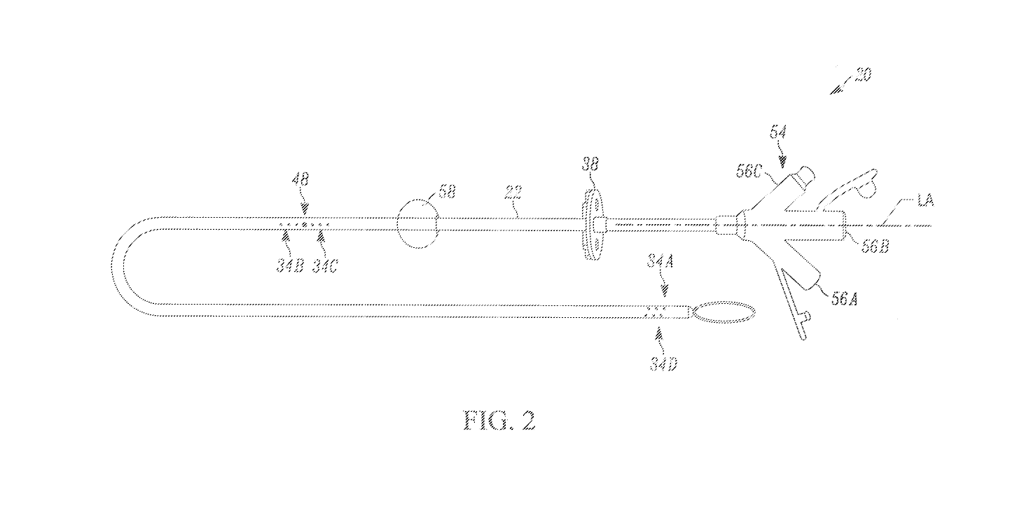

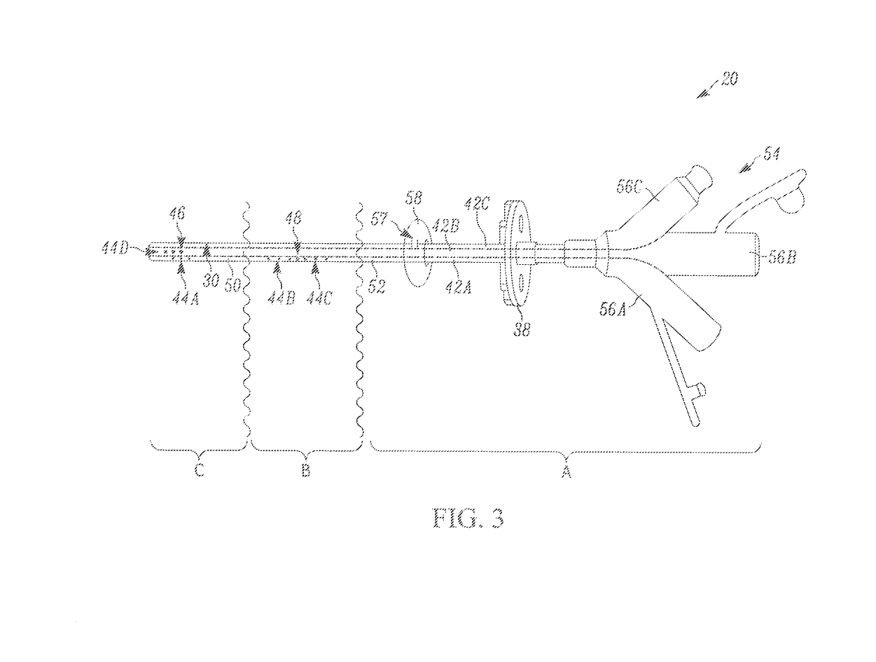

An end cap 54 can be secured to the proximal end 24 of the sheath 22. The end cap 54 can include a plurality of ports 56A-56C. In one example, the end cap 54 can include three ports 56, with a second port 56B being substantially aligned along a longitudinal axis (see FIG. 2) of the sheath 22 and a first port 56A and a third port 56C forming a Y-shape with the sheath 22. The number of ports 56 can be increased or decreased, however it is preferable for the number of the ports 56 to be equal to the number of the lumens 42. The first port 56A is in fluid communication with the second flow path 52 of the first lumen 42A, the second port 56B is in fluid communication with the second lumen 42B, and the third port 56C is fluid communication with the third lumen 42C. An inflatable device, such as a balloon 58 is secured to the outer surface 28 of the sheath 22 near the retention collar 38 toward the distal end 26 of the sheath 22. The balloon 58 is in fluid communication with the third port 56C via an outlet 57, illustrated in FIG. 3, provided on the third lumen 42C.

Each of the ports 56 can be provided with attachment features (not shown) that enable a conduit to be retained to the port 56. For example, each of the ports 56 can be provided with a Luer-Lok connection fitting. However, it will be appreciated that any other attachment features can be used (e.g., interference fit). Each of the ports 56 can further be provided with sealing members 59A-59B that close off the ports 56. In one example, the sealing members 59 can be provided with ribs that retain each sealing member 59 within the respective port 56. It is understood that any other appropriate sealing member 59 can be used.

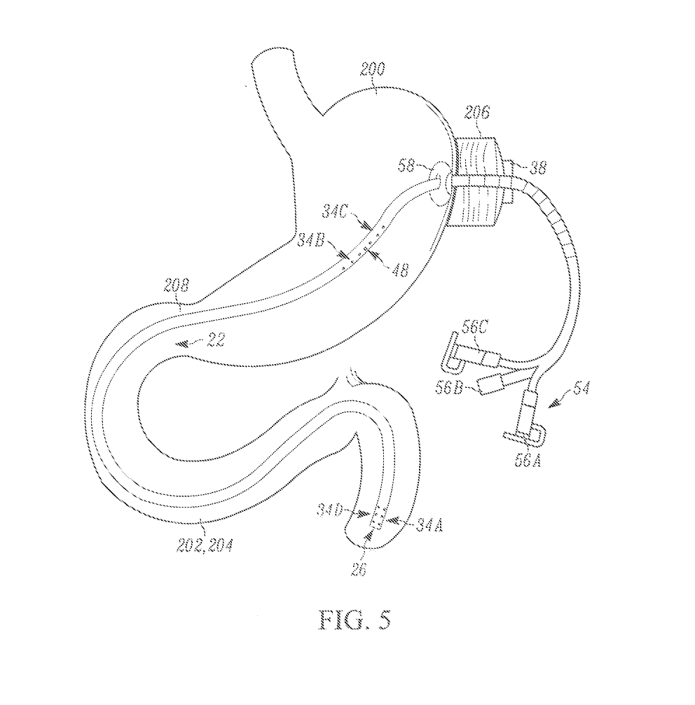

Referring to FIG. 4, a jejunal system 120 according to a second embodiment of the present disclosure is shown. The jejunal tube 120 in FIG. 4 is substantially similar to the jejunal system 20 in FIGS. 1-3 with shared features being identified by the same numeral increased by a factor of 100. That is, in the second embodiment illustrated in FIG. 4, the jejunal system 120 comprises the lumens, apertures, sheath, and other features, excepting the structure of the end cap 154, of the jejunal system 20.

As stated above, the primary change in the jejunal tube 120 in FIG. 4 is the structure of the end cap 154. The end cap 154 includes three ports 156A-156C. A second port 156B is substantially aligned with a longitudinal axis of the sheath 122, and the first port 156A and the third port 156C form a T-shape with the sheath 122. It will be appreciated that the end cap can be any suitable structure besides that which is shown in FIGS. 1-4.

### Method of Use

Referring to FIG. 5, use of the gastro-jejunal system 20 according to another example embodiment of the present disclosure will now be discussed. First, an incision is made to gain access to the interior of a patient's stomach 200. Next, the distal end 26 of the sheath 22 is inserted into the stomach 200, and the gastro-jejunal system 20 is advanced into the stomach 200 until the retention collar 38 rests against the patient's skin 206. As the gastro-jejunal system 20 is advanced, the distal end 26 of the sheath 22 is guided into the small intestine 202. An endoscope (not shown) can be used to assist guidance of the distal end 26 of the sheath 20 into the small intestine 202.

The gastro-jejunal system 20 is dimensioned and configured such that, when the retention collar 38 rests against the patient's skin 206, the second and third sheath aperture sets 34B, 34C are located in the stomach 200, and the first and fourth sheath aperture sets 34A, 34D are located in the small intestine 202 with the sheath 22 extending through the pyloric junction 208. In particular, the first and fourth sheath aperture sets 34A, 34D are located in the jejunum 204.

With the retention collar 38 resting against the patient's skin 206, the balloon 58 can be inflated to anchor the position of the gastro-jejunal system 20 within the stomach 200 and small intestine 202 of the patient. Inflation of the balloon 58 is accomplished by, first, attaching an inflation conduit (not shown) to the third port 56C. The inflation conduit can be configured to provide an inflation medium to the gastro-jejunal system 20. In one example, the inflation medium may be a fluid such as water. However, it will be appreciated that any other appropriate inflation medium can be used (e.g., air). The inflation medium, supplied from the inflation conduit, flows into the third port 56C, through the third lumen 42C, through the outlet 57, as illustrated in FIG. 3, and into the balloon 58.

The inflation conduit continues to supply the inflation medium until the balloon 58 is inflated such that the parts of the human body located between the balloon 58 and the retention collar 38 are “clamped” therebetween. This clamping anchors the position of the gastro-jejunal system 20 relative to the patient, thereby ensuring that the second and third sheath aperture sets 34B, 34C remain in the stomach 200 and the first and fourth sheath aperture sets 34A, 34D remain in the jejunum 204. The inflation conduit is removed from the third port 56C once inflation of the balloon 58 is complete.

With the gastro-jejunal system 20 and the sheath 22 positions anchored, a feeding conduit (not shown) can be attached to the second port 56B to deliver nutrients directly to the jejunum 204. Nutrients flow from the feeding conduit, into the second post 56B, and through the second lumen 42B to the fourth lumen aperture set 44D. Due to the fluid communication between the fourth sheath aperture set 34D and the fourth lumen aperture set 44D, the nutrients can flow out of the second lumen 42B and then the sheath 22 via the fourth lumen aperture set 44D and the fourth sheath aperture set 34D.

The gastro-jejunal system 20 can also be used to deliver nutrients to the stomach 200 or vent the stomach 200. If it is desired to deliver nutrients to the stomach 200, the feeding conduit can be attached to the first port 56A. Nutrients can flow from the feeding conduit, into the first port 56A, and through the second flow path 52 of the first lumen 42A to the third lumen aperture set 44C. The fluid communication between the third lumen aperture set 44C and the third sheath aperture set 34C enables nutrients to flow out of the first lumen 42A and then the sheath 20 via the third lumen aperture set 44C and the third sheath aperture set 34C. If it is desired to vent the stomach, a venting conduit (not shown) can be attached to the first port 56A. Contents to be vented from the stomach can pass through the sheath 22 and into the second flow path 52 of the first lumen 42A via the third sheath aperture set 34C and the third lumen aperture set 44C. The contents to be vented can then be removed from the stomach via the second flow path 52 of the first lumen 42A, the first port 56A, and the venting conduit.

The construction of the gastro-jejunal system 20 also allows contents of the stomach 200 to be drained into the small intestine 202. As explained above, the gastro-jejunal system 20 is dimensioned and configured to locate the second sheath aperture set 34B, (and fluidly coupled second lumen aperture set 44B) in the patient's stomach and the first sheath aperture set 34A (and fluidly coupled first lumen aperture set 44A) in the small intestine 202. Additionally, as explained above, the second lumen aperture set 44B is in fluid communication with the first lumen aperture set 44A via the first fluid flow path 50 of the first lumen 42A. Accordingly, the construction of the gastro-jejunal system 20 effectively puts the stomach 200 in fluid communication with the small intestine 202. Stomach contents or pressure can enter into the first fluid flow path 50 via the second sheath aperture set 34B and second lumen aperture set 44B, flow through the first fluid flow path 50, and into the small intestine 202 via the first lumen aperture set 44A and the first sheath aperture set 34A. Thus, the gastro-jejunal system 20 ensures that fluid communication between the stomach 200 and the small intestine 202 is not obstructed.

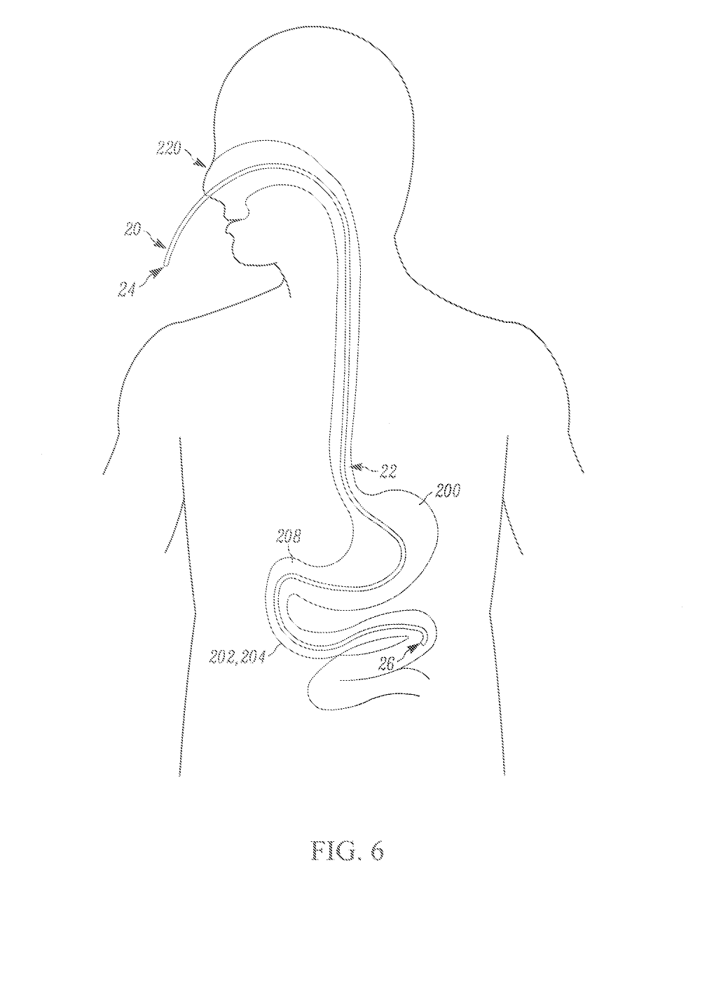

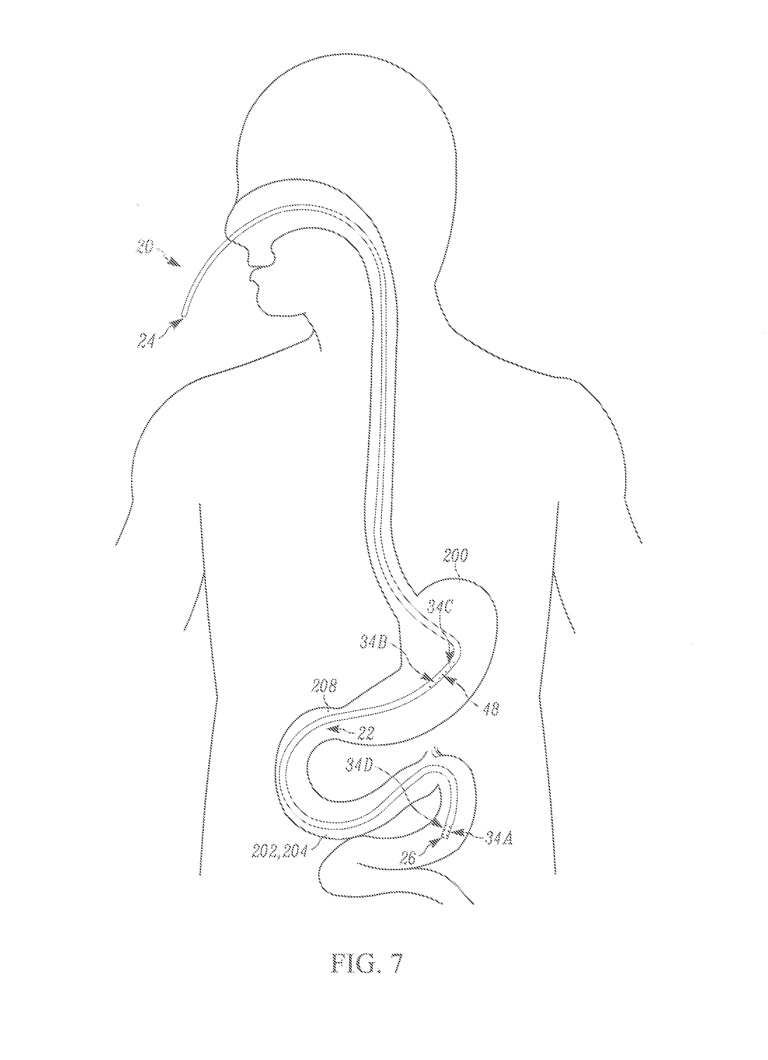

Referring to FIGS. 6-7, use of the jejunal system or apparatus 20 according to another example embodiment of the present invention will now be discussed. Instead of inserting the distal end 26 of the sheath 22 into an incision made on the patient's stomach, the distal end 26 of the sheath 22 is inserted into one of the nostrils of the patient's nose 220. This jejunal system 20 or naso-jejunal system 20 is then advanced until the second and third sheath aperture sets 34B, 34C are located in the stomach 200 and the first and fourth sheath aperture sets 34A, 34D are located in the small intestine 202 with the sheath 22 extending through the pyloric junction 208. The naso-jejunal system 20 can then function in the same fashion as discussed above in regard to FIG. 5. Of course, it is understood that the naso-jejunal system 20 in FIGS. 6 and 7 must be longer than the gastro-jejunal system 20 in FIG. 5. The proximal end 24 of the naso-jejunal system 20 may comprise an end cap (not shown), as described above with regard to the end cap 54, 154 in FIGS. 1-5. It will be appreciated that the jejunal apparatus 20 may enter the patient's body in other ways, for example, the patient's mouth.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings. For example, the apertures 36, 46 of both the sheath 22 and the lumens 44 can be replaced with a wire mesh, fluid permeable membrane, or any other suitable structure that allows fluid flow.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed.

To the extent that the materials for any of the lore going embodiments or components thereof are not specified, it is to be appreciated that suitable materials would be known by one of ordinary skill in the art for the intended purposes.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of stream lining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A jejunal system, comprising:
a sheath having a proximal end and a distal end;
a plurality of lumens extending through the sheath between the proximal end and the distal end, a first lumen of the plurality of lumens being provided with a first aperture set and a second aperture set, the first aperture set being in fluid communication with the second aperture set via the first lumen; and
wherein the jejunal system is dimensioned and configured such that, when the system is inserted into a human body, the first aperture set is located in a small intestine of the human body, and the second aperture set is located in a stomach of the human body.

2. The jejunal system of claim 1, wherein the first lumen includes a partition, the partition fluidly separating the first lumen into a first flow path and a second flow path.

3. The jejunal system of claim 2, wherein the first aperture set and the second aperture set are in fluid communication with the first flow path.

4. The jejunal system of claim 3, wherein the first lumen includes a third aperture set in fluid communication with the second flow path.

5. The jejunal system of claim 4, wherein the jejunal system is dimensioned and configured such that, when the system is inserted into the human body, the third aperture set is located in the stomach.

6. The jejunal system of claim 4, wherein the third aperture set is fluidly divorced from the first aperture set and the second aperture set.

7. The jejunal system of claim 1, wherein a second lumen, of the plurality of lumens, is provided with a fourth aperture set.

8. The jejunal system of claim 7, wherein the jejunal system is dimensioned and configured such that, when the system is inserted into the human body, the fourth aperture set is located in the small intestine.

9. The jejunal system of claim 1, wherein an end cap, secured to the proximal end of the sheath, comprises a plurality of ports in fluid communication with the plurality of lumens.

10. The jejunal system of claim 9, wherein a first port, of the plurality of ports, is in fluid communication with the first lumen, a second port, of the plurality of ports, is in fluid communication with a second lumen, of the plurality of lumens, and a third port, of the plurality of ports, is in fluid communication with a third lumen, of the plurality of lumens.

11. The jejunal system of claim 10, wherein the sheath comprises a balloon and a retention collar secured to the sheath, the retention collar located between the balloon and the end cap, wherein the third port is in fluid communication with the balloon.

12. A method of forming a jejunal system comprising:
advancing a plurality of lumens through a sheath, the plurality of lumens running between a proximal end of the sheath and a distal end of the sheath;
positioning a first aperture set and a second aperture set in a first lumen of the plurality of lumens, wherein the first aperture set is in fluid communication with the second aperture set via the first lumen; and
dimensioning and configuring the jejunal system such that, when the system is inserted into a human body, the first aperture set is located in a small intestine of the human body, and the second aperture set is located in a stomach of the human body.

13. The jejunal method of claim 12, comprising positioning a partition within said first lumen, the partition fluidly separating the first lumen into a first flow path and a second flow path, wherein the first aperture set and the second aperture set are in fluid communication with the first flow path.

14. The jejunal method of claim 13, comprising:
positioning a third aperture set in the first lumen such that the third aperture set is in fluid communication with the second flow path; and
dimensioning and configuring the jejunal system such that when the system is inserted into the human body, the third aperture set is located in the stomach.

15. The jejunal method of claim 12, comprising:
forming a fourth aperture set in a second lumen comprised in the plurality of lumens; and
dimensioning and configuring the jejunal system such that, when the system is inserted into the human body, the fourth aperture set is located in the small intestine.

16. A jejunal system, comprising:
a sheath having a proximal end and a distal end;
a plurality of lumens extending through the sheath between the proximal end and the distal end, the plurality of lumens comprising:
a first lumen being provided with a first aperture set, a second aperture set and a third aperture set, the first lumen comprising a partition fluidly separating the first lumen into a first flow path and a second flow path, wherein the first aperture set and the second aperture set are in fluid communication with the first flow path, and the third aperture set is in fluid communication with the second flow path; and
a second lumen being provided with a fourth aperture set; and
wherein the jejunal system is dimensioned and configured such that, when the system is inserted into a human body, the first aperture set and the fourth aperture set are located in a small intestine of the human body, and the second aperture set and the third aperture set are located in a stomach of the human body.

17. The jejunal system of claim 16, wherein the third aperture set is fluidly isolated from the first aperture set and the second aperture set.

18. The jejunal system of claim 16, wherein an end cap, secured to the proximal end of the sheath, comprises a plurality of ports in fluid communication with the plurality of lumens.

19. The jejunal system of claim 18, wherein a first port, of the plurality of ports, is in fluid communication with the first lumen, a second port, of the plurality of ports, is in fluid communication with the second lumen, and a third port, of the plurality of ports, is in fluid communication with a third lumen, of the plurality of lumens.

20. The jejunal system of claim 19, wherein the sheath comprises a balloon and a retention collar secured to the sheath, the retention collar located between the balloon and the end cap, wherein the third port is in fluid communication with the balloon.
